(19) United States
(12) Patent Application Publication
(10) Pub. No.: US 2009/0104643 A1
(43) Pub. Date: Apr. 23, 2009

(54) BIOLUMINESCENCE-BASED SENSOR WITH CENTRIFUGAL SEPARATION AND ENHANCED LIGHT COLLECTION

(76) Inventor: Daniel A. Bartholomeusz, Poway, CA (US)

Correspondence Address: Workman Nydegger;1000 Eagle Gate Tower, 60 East South Temple, Salt Lake City, UT, 84111, US

(21) Appl. No.: 12/067,002

(22) PCT Filed: Sep. 13, 2006
(86) PCT No.: PCT/US06/35639

§ 371 Date: Sep. 8, 2008

Related U.S. Application Data
(60) Provisional application No. 60/717,795, filed on Sep. 15, 2005.

(51) Int. Cl. C12Q 1/02; C12M 1/00

(52) U.S. Cl. 435/29; 435/288.7

(57) ABSTRACT

In general, embodiments of the present invention relate to a bioluminescence-based point of care device that is made up of at least one reaction well (89) that contains a bioluminescent reagent for a luminescent reaction, sample well (80), sample collection well (84), and reagent well (87). A sample is introduced into the reaction wells (89), where it dissolves the reagents and initiates the luminescent reaction, where a luminescence signal is then transmitted through a window to a photo detector.

### PRIORITY CLAIM

This application claims the benefit, under 35 U.S.C. § 119(e), of the filing date of U.S. Provisional Patent Application Ser. No. 60/717,795, filed Sep. 15, 2005, for “BIOLUMINESCENCE-BASED BLOOD SENSOR WITH CENTRIFUGAL PLASMA SEPARATION, LOW TEMPERATURE ENZYME PACKAGING, AND ENHANCED LIGHT COLLECTION”, the contents of which are incorporated by this reference.

### GOVERNMENT LICENSE RIGHTS

The work underlying this sensor was paid for, in part, by NIH RFP#PAR01-057, Project#1R21RR17329 awarded by the National Institute for Health, Technology Development for Biomedical Applications Grant. The U.S. government may have certain rights to this invention.

### TECHNICAL FIELD

Embodiments of the present invention generally relate to point-of-care (POC) luminescent sensors, such as for use with diagnostics.

### BACKGROUND

Point-of-Care biosensors are generally clinical quality, analytical devices used for in vitro diagnostics (“IVD”). Various of these devices have been recognized to have improved healthcare by operating where treatment decisions are made, or at the point-of-care (“POC”). Suitable, non-limiting examples of POC locales include the emergency room, outpatient clinics, nursing homes, alternative-care centers, a patient's home, a hospital bedside, a battlefield, a campsite, and/or the like. Generally, any location where a need exits to measure and/or monitor a sample can be a POC.

IVD device manufacturers use a variety of miniaturization technologies in order to cost-effectively bring clinical chemistry lab results to the point-of-care. There are review articles that cover the microfabrication technologies as they apply to biosensing applications. {1, 2, 3} These microfabricated sensors, or micro-Total Analysis Systems (μTAS), integrate sample preparation, fluid handling, chemical sensing components, and detection systems all on the same device. Various semiconductor fabrication methods have been used to miniaturize the detection systems as well couple them to the miniaturized analytical platforms. Other technologies that have made smaller POC devices possible include smaller and faster computers, electronics, and interactive screens. {4}

μTAS merge various microfabrication technologies with analytical chemistry platforms to miniaturize the core sensing technologies. Microfluidic fabrication technologies enable the devices to use smaller amounts of reagent and sample for performing the actual measurements. In many cases, the reduction in size improves the detection limits. Miniaturized POC devices are able to measure routine clinical chemistry assays existing in micromolar to millimolar range from picoliter to microliter sample volumes.

Most POC sensors perform tests on whole blood samples that are less than 100 μL (2), while others use blood preparations such as plasma, urine, saliva, or expired gases. Despite this possibility, many clinical assays still require milliliter sample volumes because their sensors are not used at the point-of-care and extra sample volume is used for transporting to the lab. Miniaturization technologies bring the testing to the point-of-care and reduce the cost per test as well as improve patient comfort. These technologies also reduce the size and cost of POC devices, making them practical to use in a POC setting.

Given the potential they have for reducing healthcare costs, POC devices have become a multibillion dollar market, and are a fast growing sector within IVD testing. {4) In 1998, POC sales in the US were at $2.4 billion and were projected to be at $4 billion by 2003. {5} Manufacturers attempting to enter this market must make their products cost effective for the end-user in the appropriate market application and have a stable core technology for a functional product. {4}

Many POC device manufacturers base their initial products on a few core sensing technologies, which are often different proprietary chemistries, before expanding their measurement capabilities. {4) Most focus their measurements on specific market applications, such as measuring glucose for handling diabetes. Savings per test can be compounded by incorporating multiple assays on the same device, giving more data for the physician or other health care provider to work with per dollar spent. In order to achieve this, manufacturers often use modular cartridges that use the same detection system in the POC device. Cartridges with new test panels are developed later to address other market applications.

A POC device with a broad range of sensing capabilities would allow many analytes to be measured for a variety of applications. Such a device would make it practical to measure multiple analytes in basic and clinical research, personal disease management, or clinical and hospital use. Improved practicality to measure multiple metabolites at the point-of-care would further increase the demand for understanding the complex relationships between diseases and their manifestation in the metabolic domain. Comprehensive metabolic diagnostic panels could be customized using existing knowledge of how certain diseases are manifested in abnormal metabolite concentrations. One example would be a low cost comprehensive inborn metabolic error diagnostic panel that can identify many disorders such as phenylketonuria (PKU) or galactosemia. Other panels can be developed as the complex metabolic relationships are discovered for certain diseases. This device could aid in collecting data for metabolic modeling, which will lead to understanding the complex relationships between diseases and metabolite concentrations.

Measuring and/or monitoring is typically performed by a sensor that measures various analytes such as electrolytes (for example, and not by way of limitation, Na+, K+, etc.), chemistry (for example, and not by way of limitation, glucose, lactate, blood gases, pH, metabolites, etc.), blood characteristics (for example, and not by way of limitation, hemoglobin, prothrombin time, etc.), as well as steroids, drugs, viruses, and/or the like. {4, 5}

In the healthcare industry, prior POC biosensors have been able to measure such analytes from small samples, usually blood or urine, within minutes, providing quick information needed for caregivers to make decisions when diagnosing or monitoring a patient's condition. It is generally accepted that rapid measurements lead to more effective patient visits, shorter hospital stays, and improved diagnostics. POC devices have been credited with allowing patients to manage and/or monitor their conditions away from the hospital.

POC devices help address analytical performance requirements, compliance issues, and rising healthcare costs by performing the tasks of centralized chemistry labs. Central chemistry labs may be within or off-site from the hospital and can take anywhere from 4 to 72 hours for measurement results. Due to costs associated with these labs, government and insurance companies have driven hospitals to reduce the amount of lab tests performed. Unfortunately, such actions come at the expense of patients' health. To address clinical chemistry needs, POC devices are being designed to perform a menu of 70 routine tests which cover about 90% of care center needs. To be effective, POC devices must be designed to be the lowest cost per reportable result. Each POC test provides about 35% cost saving per analysis and additional savings in manpower. {6} POC devices save time by provide rapid results, often in less than 30 minutes.

Luminescence-based analysis is a highly specific and sensitive analytical method. The specificity of luminescence-based analysis is determined by specific reactions that couple analytes to a luminescent reaction, which produces light proportional to the analyte concentration. Bioluminescence-based analysis is a specific type of luminescence-based analytical method involving enzymatic reactions coupled to an enzyme-based luminescent. The specificity of the reaction for the metabolite or analyte of interest is determined by the enzyme coupling reaction. The inherently sensitivity of luminescence-based analysis is due to the high quantum efficiency, which can be up to 90% for bioluminescent reactions, and the low background noise. Efficient light emission with low background coupled with the high sensitivity allows luminescence to be up to 100 to 1,000 times more sensitive than fluorescence. Luminescence does not require the filters and sources associated with fluorescence-based analysis. Luminescence background comes from nonspecific interactions of the non-luminescent coupling reaction and nonspecific light emission of the chemiluminescent molecule. This nonspecific light emission is caused by unwanted oxidants, metal catalysts, pH differences, enzymatic activity, and other variables. Thermal degradation is another mode of unwanted light emission and is specific for the chemiluminescent label or analyte being measured. Another attracting characteristic of luminescence-based assays is that they have a detection range of five or more orders of magnitude without dilution or concentration of the sample fluid. The dynamic range characteristic is due to the high signal to noise ratio intrinsic to luminescence measurements and also because of the ability to “tune” the dynamic range via modulation of enzyme activity and/or enzyme type.

Luminescence detectors and/or sensors have not yet found great commercial applicability in the POC market. Currently, commercially available luminescent detection systems are mainly used in the laboratory for measuring single analytes in trace amounts. These systems are generally PMT-based luminometers that measure single samples or multi-well plates with volumes greater than 25 μL. Such detection systems are available from Bio-Rad (Hercules, Calif.), Berthold Detection Systems GmbH (Oakridge, Tenn.), Turner Designs (Sunnyvale, Calif.). As well, handheld luminometers used for detecting biomass and bacterial contamination from swabbed samples are known in the art.

A factor influencing why bioluminescence has not been readily applied to POC applications is a perception that luciferases and other reagents involved are somewhat labile, unstable, and difficult to utilize, with precise and somewhat sophisticated protocols. However, recent advances in enzyme stabilization techniques have produced highly active, thermally stable mutant luciferases have become available. {7;8;9} Although, despite the advances, there is limited work in packaging bioluminescent assays in microfluidic devices.

Most luminescent assays on microfluidic structures involve chemiluminescence. Examples involving chemiluminescence-based assays in microfluidic systems are used for a variety of biosensing applications. Single analyte chemiluminescent assays in liquid form have been performed in microfluidic channels. Chemiluminescent and bioluminescent immunoassays have been used and even on a chip {10} to measure drug levels and for detecting cancer markers.

The work to date involving chemiluminescence-based assays in a microfluidic systems are used for a variety of biosensing applications. Single analyte chemiluminescent assays in liquid form have been performed in microfluidic channels. {19} Chemiluminescent and bioluminescent immunoassays have been developed {20} to measure things like drugs and cancer marker. Others have started to package chemiluminescent reagents in microfluidic structures for potential POC applications. There is even one implantable glucose device using immobilized glucose oxidase for a chemiluminescent reaction in a flow-through sampling device that has been tested.{12} Currently, these examples mix reagents with the sample via merging microfluidic channels to measure one analyte with a single PMT downstream. A low cost luminometer for measuring a single analyte from luminescent reactions has been tested using a photodiode and a transimpedance op-amp circuit. {12}

Other detectors include an implantable glucose device using immobilized glucose oxidase for a chemiluminescent reaction in a flow-through sampling device that has been tested. {11} Currently, these examples mix reagents with the sample via merging microfluidic channels to measure one analyte with a single PMT downstream. A low cost luminometer for measuring a single analyte from luminescent reactions has been tested using a photodiode and a transimpedance op-amp circuit. {12}

POC devices use detection systems to measure physical, electrical, thermal, or optical stimuli as a function of some chemical interaction of an analyte with the sensing system. {1, 2, 4}

How analytical methods have been implemented in μTAS and POC devices, along with a description of their applications can be found in references {6}, {17}, and {18}. Table 1-2 shows the concentrations ranges of general metabolites of interest for POC applications. Table 1-3 shows examples of some POC devices and the number of analytes that can be measured from the same sample for each device. The example analytes listed are ones tested in this research as will be described later.

Detector cost and size is another determinate in developing a multi-analyte bioluminescence-based sensor. Most luminometers use photo-multiplier tubes (PMT) due to their high sensitivity, however, due to their large size they have not been extensively used in microfluidic multi-analytes devices. Comparably sensitive CCDs can be used for measuring multiple luminescent reactions in parallel, but are too expensive for POC applications.

Microfabrication techniques have been used by some researchers to address some of the detection issues associated with microfluidic luminescent reactions. Complimentary metal oxide semiconductor (“CMOS”) integrated circuits have been used to detect bioluminescent signals for whole-cell monitoring, nucleic acid, protein, and pathogen detection. These integrated systems are able to measure the light signal as well as perform signal conditioning and auxiliary functions such as calibration. Although the CMOS detectors are not as sensitive as PMT detectors, they are able to integrate signals. Their custom configurations have also allowed for close contact (high collection angle) optical coupling which improves their detection limits compared to standard image collection optical coupling. Other researchers used fused glass microchannels to improve light output for enzyme catalyzed chemiluminescence assays. Instead of using single, large wells, multiple glass capillary channels are fused together, increasing the surface area for which the enzymes can be immobilized to, thus increases the luminescent reaction rate and yields greater light intensities.

In 1990, the concept of μTAS devices and the potential miniaturization has for certain chemical sensing applications was published in Manz A et al., “Miniaturized Total Chemical Analysis Systems: A Novel Concept for Chemical Sensing,” Sensors and Actuators, B1(1-6):244-248, 1990. In Manz, the minimum detectable analyte concentration (“Cmin”) was stated as being strictly inversely proportional to the sample volume V, as determined by the detection limit (“Dn”), (in moles) of the sensor. This relationship, however, does not show how the scaling effects of miniaturization can actually improve the detection limit.

Although bioluminescence-based analysis is well known and has been used regularly in research, it has not been widely applied to POC or routine clinical analysis. Specifically, a luminescence-based device has not been created for measuring multiple analytes at the point-of-care. Also, such a device has not been created with sample preparation functionality (blood and plasma separation). Multiple luminescence-based assays have not been packaged on a POC device in stable form in volumes less that 1 μL. Also, the ability to aliquot small sample volumes (less that 1 μL) to multiple reaction wells for measuring different analytes has not been implemented in a POC device. The sensitivity and broad measurement capabilities of bioluminescence-based analysis allows multiple analytes to be measured from the same sample; even, for example, capable of measuring 100 different analytes from a sample fluid as small as 100 μL.

There do exist in the art, methods for handling sample volumes less than 100 μL. One such sample delivery method is centrifugal pumping. Centrifugal pumping is an ideal sample delivery method for the proposed bioluminescence-based device. It is based on using centrifugal force to move fluids radial outward from the center of a disk with fluidic channels. Centrifugal pumping is capable of valving, decanting, calibration, mixing, metering, sample splitting, separation, and capillarity without sensitivity to bubbles, ions, or type of fluid. Centrifugal sample delivery and processing system has been shown to produce significant advantages and have been used for POC applications. Centrifugal systems have been used in clinical chemistry applications since the 1970's.{15} Initially the devices were injection molded in plastic and used sample volumes greater than 100 μL. In the early 1990s, a rotary analyzer that used less than 100 μL of blood was reported.{13, 14} In 1998 Madou and Kellogg introduced a microfabricated centrifugal device on a CD.{16} However, bioluminescence-based analysis has not been implemented on a centrifugal-based sample delivery system for POC applications.

Recent prior art uses centrifugation on a CD device to separate plasma. {21} Sample metering or aliquoting has also been used on a CD platform for high surface tension fluids such a water, using hydrophobic passive valves. {22} These passive valves hold fluids in pace until the CD is spun at higher frequencies where the centrifugal force pushed the fluid past the hydrophobic barrier. However, aliquoting plasma to multiple sections has not been developed on a CD type device due to the low surface tension of plasma, which tends to burst past the passive valves of current art, at low spinning speeds. Because of the problems with metering plasma by prior art passive valve, plasma separation and sample metering have not been combined on the same device.

However, the art field has not incorporated a photodetector and microfabricated centrifugal device. Accordingly, the art field is in need of a POC device with the sensitivity of a luminescence-based detector.

Accordingly, the art is in need of various embodiments of systems that are designed with one or more of the following considerations: various systems were developed with a sensitivity to measure analyte concentrations to be measured from volumes less than 1 μL per analyte allowing potentially hundreds of analytes to be measured from the same sample volume, to the development and/or use of thermally stable enzymes and enzyme stabilization techniques, room temperature sealing of microfluidic devices, via “Xurography”, and/or other related methods, the ability to aliquot volumes less that 1 μL from a single sample to multiple reaction wells for measuring multiple analytes; sample integration (for example, and not by way of limitation, plasma separation) on the same device; the use of parallel or serial sample delivery via sample wicking membranes or centrifugal microfluidic pumping; unique passive valves that can handle low surface tension fluids such as plasma and perform sample metering; sequential mixing of stabilized reagents for specific assays systems; and/or the like.

### DISCLOSURE OF INVENTION

In general, embodiments of the present invention relate to a luminescent-based micro-total analysis system (μTAS), platforms, and related methods. Various embodiments of the invention are capable of measuring multiple analytes of a sample. Alternative embodiments comprise luminescence-based assays on a multi-analyte POC device. In various embodiments, the POC device or platform comprises channel means into which a sample is introduced. The channel means, in varying embodiments, contain reagents. The reagents may be added to the channels or be stored in the channel after fabrication in a stabilized form.

A sample introduced into the channel means, dissolves the reagents, and initiates a luminescent reaction. The luminescence is then transmitted through a window or aperture to a photo detector. Further embodiments comprise multiple detectors for detecting a luminescence from multiple reaction wells. Various configurations of the reaction wells of the present invention allow for series and/or parallel processing of samples. Further embodiments comprise an on board calibration function.

### BRIEF DESCRIPTION OF THE DRAWINGS

FIG. 1 is an illustration of an embodiment of the present invention.

FIG. 2 is an illustration of a side perspective of the embodiment of FIG. 1.

FIG. 3 is an illustration of an alternative embodiment of a platform of the present invention from a perspective above the platform.

FIG. 4 is an illustration of an alternative embodiment of the present invention.

FIG. 5 is an illustration of an experiment performed in an embodiment of FIG. 4.

### BEST MODE(S) FOR CARRYING OUT THE INVENTION

As used herein, the term “luminescence” means and refers to the production of visible light by a chemical reaction or reactions.

As used herein, the term “bioluminescence” means and refers to the production of light by a chemical reaction via an enzyme.

As used herein, the term “soft-lithography” means and refers to a technique developed to allow for the rapid prototyping of, for example, microfluidic devices.

As used herein, the term a “photoresist(s)” means and refers to a light sensitive material used in the process of photolithography to form a patterned coating on a surface. As used herein, photoresists are classified into two groups, positive resists, in which the exposed areas become more sensitive to chemical etching and are removed in the developing process, and negative resists, in which the exposed areas become resistant to chemical etching, so the unexposed areas are removed during the developing process.

In general, embodiments of the present invention relate to a bioluminescent-based micro-total analysis system (μTAS), platforms, and related methods. Various embodiments of the invention are capable of measuring multiple analytes from a sample. Further embodiments of the present invention comprise a bioluminescence-based analyte or multi-analyte POC device. In various embodiments, the POC device or platform comprises a reaction means. In embodiments, the reaction means comprises a suitable channel means comprising reaction/reagent well(s), line(s)/channel(s), valve(s), waste container(s), vent(s), and/or the like into which a sample is introduced. The suitable reaction/reagent well(s), line(s)/channel(s), valve(s), waste container(s), vent(s), and/or the like, in varying embodiments, contain reagents, such as luminescent reagents or bioluminescent reagents. The reagents may be added to the channel means or be stored in the channel means after formation or fabrication, optionally, in a stabilized form.

A sample introduced to the channel means dissolves the reagents and initiates a luminescent reaction. In one embodiment, the reagent is dissolved in a reagent well and/or reaction well. A luminescence from the reaction is then transmitted through a window to a photo detector. Further embodiments comprise multiple detectors for detecting a luminescence from multiple reaction wells. Various configurations of the reaction wells of the present invention allow for series and/or parallel processing of samples.

In an embodiment of a platform of the present invention, suitable reaction well(s), line(s)/channel(s), valve(s), waste container(s), vent(s), and/or the like are made on the platform by coating photoresist, such as an epoxy-based photoresist, on a compact disk (CD) wafer. However, any suitable photoresist may be used. In various embodiments, the photoresist is then selectively cross-inked by photopolymerizing the resist by using a mask, such as in an ultraviolet light (UV) treatment. Suitable methods and materials for forming a photo resist mask can be found in U.S. Pat. Nos. 6,689,541; 6,673,721; 6,660,645; 6,593,039; 6,451,511; 6,340,603; 6,329,294; 6,200,884; 6,121,154; 6,063,695; 6,025,268; 5,980,768; 5,918,141; 5,902,704; 5,677,242; 5,667,940; 5,290,713; 5,015,595; and, 4,341,571, the contents of all of which are hereby incorporated by reference in their entirety. Unexposed photoresist is then washed away, thereby forming a suitable reaction/reagent well(s), line(s)/channel(s), valve(s), waste container(s), vent(s), and/or the like.

In an alternative embodiment, the suitable reaction well(s), line(s)/channel(s), valve(s), waste container(s), vent(s), and/or the like are made by injection molding. In alternative embodiments, the suitable reaction well(s), line(s)/channel(s), valve(s), waste container(s), vent(s), and/or the like are made by successive layers of a thermoplastic, adhesive films, heat stackable films, or other construction material.

In a method of fabrication, a base means is immobilized, such as by placing in a plastic container, clamping, and/or the like. A suitable hardening mixture, a matrix, is applied to the base, such as, and without limitation, a siloxane or a vinyl, over the surface. The matrix is then formed to create a suitable reaction/reagent well(s), line(s)/channel(s), valve(s), waste container(s), vent(s), and/or the like. In particular embodiments, the forming is by cutting, folding, slicing, drying, removing, dissolving, and/or the like.

In particular embodiments, a suitable base means is a CD, such as a translucent CD or metalized CD. In an alternative embodiment, a suitable base is a glass slide. In an alternative embodiment, a suitable base is a clear plastic sheet. However, suitable platforms may generally be any structure with a surface to accept a cover, such as a plate, a gel, a glass sheet, and/or the like. Other embodiments are formed without the assistance of a base, such as when the cover is formed directly upon a window.

The amount of hardening material applied to the wafer may vary according to the desired matrix depth sought. In particular embodiments, the depth is between about 1 microns to about 2 cm. In an alternative embodiment, the depth is between about 0.1 mm to about 1 cm. In an alternative embodiment, the depth is between about 0.5 mm to about 0.5 cm. In an alternative embodiment, the depth is between about 1.0 mm to about 0.1 cm. In an alternative embodiment, the depth is between about 1.5 microns to about 500 microns. In an alternative embodiment, the depth is between about 5 microns to about 250 microns. In an alternative embodiment, the depth is between about 10 microns to about 100 microns.

The cutting may be performed by any process a suitable reaction/reagent well(s), line(s)/channel(s), valve(s), waste container(s), vent(s), and/or the like. In particular embodiments, the suitable reaction/reagent well(s), line(s)/channel(s), valve(s), waste container(s), vent(s), and/or the like are cut out with a knife plotter. In an alternative embodiment, a suitable reaction/reagent well(s), line(s)/channel(s), valve(s), waste container(s), vent(s), and/or the like is made by laser cutting. Suitable examples of a laser cutting process comprise polymeric fabrication processes available through Micronics Inc. (Redmond, Wash.). In an alternative embodiment, a suitable reaction/reagent well(s), line(s)/channel(s), valve(s), waste container(s), vent(s), and/or the like is made by cutting the cover. The cutting of the channels may be performed manually, automatically, and/or with the assistance of a machine. In general, any method may be used to form a suitable reaction/reagent well(s), line(s)/channel(s), valve(s), waste container(s), vent(s), and/or the like within the cover. In an alternative embodiment, xurography is used to form the channels and layer or align multiple layers of microchannels cut in adhesive backed polymeric films. Embodiments of xurography are disclosed in U.S. provisional application 60/669,570, titled Rapid prototyping of micro-structures using a cutting plotter, filed Apr. 8, 2005.

The width of the cut will be ideally suited for the particular reagents and/or sample to be tested. In particular embodiments, the width of a cut is between about 1 μm to about 2 mm. In an alternative embodiment, the width of a cut is between about 5 μm to about 500 μm. In an alternative embodiment, the width of a cut is between about 10 μm to about 100 μm. In particular embodiments, the width of a cut is between about 25 microns to about 50 microns. In various embodiments, heating the cover after application to a glass slide will increase the width of a cut.

Drying and/or hardening the cover is performed as appropriate for the particular cover. For a typical embodiment comprising a siloxane, hardening occurs naturally and can be accelerated or initiated at an elevated temperature. After drying and/or hardening, in particular embodiments, the cover is removed from the platform, if used. In various embodiments, entrance and/or exit holes for any reaction, carrier and/or sample fluids are formed. In embodiments, to immobilize the cover, the cover is applied to a glass slide.

In particular embodiments, the suitable reaction/reagent well(s), line(s)/channel(s), valve(s), waste container(s), vent(s), and/or the like are cut or cast to a depth in a matrix of about the width of the cover so that the suitable reaction/reagent well(s), line(s)/channel(s), valve(s), waste container(s), vent(s), and/or the like are adjacent the base means. In particular embodiments, the depth is completely through the cover. In an alternative embodiment, the depth is about through the cover. Depths at about the width of the cover lessen any interference for measuring luminescence through the glass slide and into the suitable reaction/reagent well(s), line(s)/channel(s), valve(s), waste container(s), vent(s), and/or the like. In particular embodiments, the luminescence is measured in the reaction well. In particular embodiments, the luminescence measured is a bioluminescence.

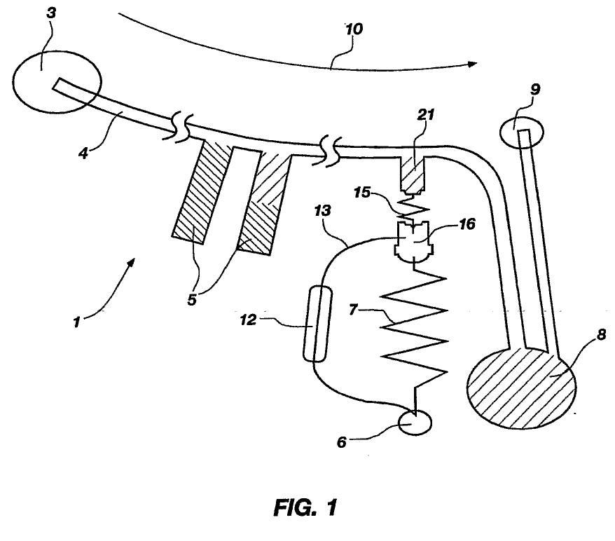

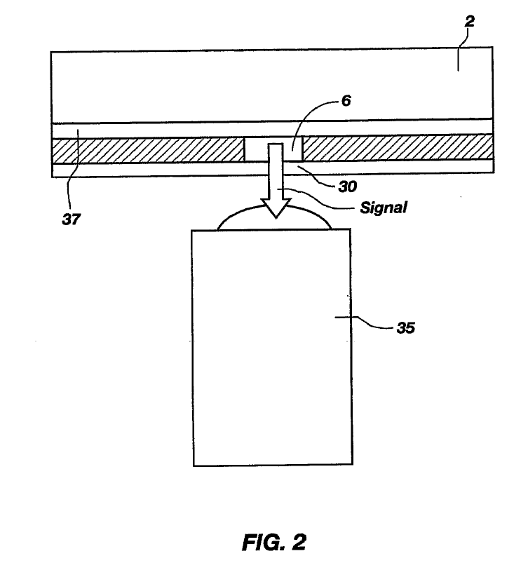

Now referring to FIG. 1, FIG. 1 is a view of a portion of a platform 1 onto which a matrix 22 (illustrated in FIG. 2) and a cover 20 (also illustrated in FIG. 2) has been applied. In this embodiment, matrix 22 comprises cuts to form an input sample well 3, a line 4, a decant chamber(s) 5, a sample collection well 21, a sample metering valve 15, a reagent well 16, a vent(s) 12, a reagent valve 18, a waste container 8, and an exit port 9.

In various embodiments, reagent well 16 comprises reagents for a luminescent reaction with the sample. In various embodiments, the reagents are for a bioluminescent reaction. The reagents may be lyophilized, liquid, solid, and/or the like. Further, the reagents may be loaded in the reaction well at or about the time of the introduction of the sample or after cutting of the reagent well. Alternative embodiments comprise a step of loading a lyophilized reagent(s) into the reagent well at the time of cutting the reagent well. The preloading of reagent allows for storage of the platform so that it may be “used off the shelf.” The reagent may be stabilized to allow for a longer duration of storage prior to use.

In an embodiment as illustrated in FIG. 1, a sample is introduced through and/or into sample well 3. A motor 40 (illustrated in FIG. 2) or other means rotates platform 1 in the direction of the rotation arrow 10, in this embodiment, in a counter clockwise direction. Motor 40 can be a compact disk drive, a modified compact disk drive, or any other motor capable of rotating platform 1 in a suitable manner. In various embodiments, motor 40 is capable of rotating from about 1 Hertz (Hz) to about 1000 Hz. In an alternative embodiment, motor 40 is capable of frequencies from about 5 Hz to about 100 Hz. In an alternative embodiment, motor 40 is capable of frequencies from about 10 Hz to about 75 Hz. However, a suitable rotation speed may be chosen and an appropriate motor 40 selected for any desired speed of rotation of frequency.

The sample may be introduced manually by a user, mechanically by a sampling device, or any other method common in the art.

It is known in the art that the rotation of platform 1 will cause a centrifugal force on platform 1. The centrifugal force will tend to be directed away from about the center of platform 1. As platform 1 is rotated faster, the centrifugal force increases.

The centrifugal force on platform 1 enables the sample to traverse line 4, past decant chamber(s) 5, through sample well 21, across sample metering valve 15, and into reagent well 16. The introduction of sample into reagent well 16 will tend to wash a reagent in reagent well 16 past reagent valve 18 and into reaction well 6.

Generally, samples may be of any form or state. In particular embodiments, the sample comprises water and/or is aqueous based. In an alternative embodiment, the sample comprises urea. In an alternative embodiment, the sample comprises blood. In alternative embodiment, the sample comprises urea. In an alternative embodiment, the sample comprises another biological fluid. However, embodiments of the present invention are not limited to particular samples.

In various embodiments, valve 15, valve 7, line 4, and/or well 16 is hydrophobic. Hydrophobicity can be used to assist in controlling the flow of sample.

In particular embodiments, the sample and the reagent begin reacting upon contact. In various embodiments, only a measured or certain amount of sample is allowed to pass valve 15. Any remainder passes to waste container 8 and/or other sample well(s) 21. Various embodiments remove waste through port 9. Waste may be removed by suction, by further rotation, by mechanical means, and/or the like.

Now referring to FIG. 2, an illustration of a side perspective of FIG. 1, the orientation of glass slide 30 and cover 2 is made apparent. In particular embodiments, reaction well 6 is adjacent glass slide 30. A detector 35 or multiple detectors 35 are positioned below slide 30 to detect and/or measure the luminescence from reaction well 6, through slide 30, as is indicated by the arrow representing a signal.

In various embodiments, a reflective metal coating 37 is applied within platform 1. In particular embodiments, coating 37 is applied above reaction well 6. Coating 37 acts to increase the reflectance and signal strength of a reaction in reaction well 6.

In various embodiments, sample delivery and detection are in series, parallel, or a combination of the two. The choice between serial and parallel detection depends on the type of detector and/or the type of application/measurement. An array of photo detectors (CCD, photodiode array, CMOS, and/or the like) enables parallel measurement from multiple wells. An alternative embodiment is a single detector that can be repositioned relative to each reaction well fast enough to measure frequency components of the bioluminescent signals. In such an embodiment, a sample can be delivered in series to each reaction well. However, other photo detectors will be apparent to those of ordinary skill in the art.

Bioluminescent-based chemical analysis is a specific type of luminescence which involves an enzyme in luminescent reaction. Two bioluminescent-based platform reactions that are used to measure a wide range of metabolites with platforms of the present invention comprise ATP (Adenosine Triphosphate) and NADH (nicotinamide adenine dinucleotide), the energy currencies of biology. Since most metabolites in the body are within one or two enzymatic reactions from ATP or NADH, they can be measured by coupling the appropriate enzyme reaction(s) to an ATP or NADH bioluminescent reaction and measuring the light output. During the production or consumption of a metabolite of interest, enzyme linked reactions will cause the production or consumption of ATP (or NADH) through the bioluminescent platform reactions shown below.

In general, the ATP reaction is based on the following firefly luciferase (FL)

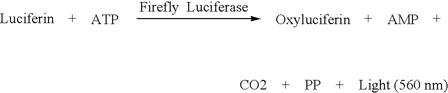

The NADH reaction is based on the following NADH:FMN oxidoreductase (OR) and bacterial luciferase (BL):

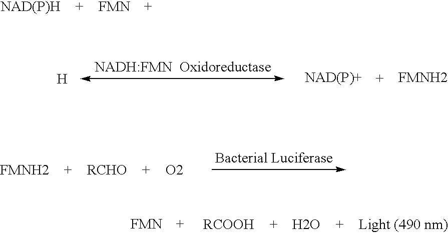

Substrates are coupled to the ATP or NADH reactions through the following generic reaction:

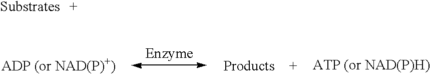

Appropriate enzymes can then be placed in the suitable reaction well(s), line(s)/channel(s), valve(s), waste container(s), vent(s), and/or the like to facilitate one of the above luminescent reactions. Further embodiments comprise applying oxygen plasma to the cover which oxidizes the bioluminescent enzymes and reagents in place on the platform.

In addition to these bioluminescent reactions, a similar and suitable chemiluminescent reaction involving hydrogen peroxide (H2O2) is available:

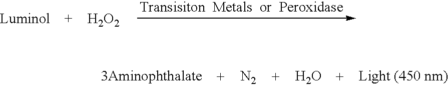

Substrates are coupled to the reaction through the following generic reaction:

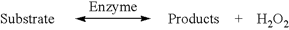

Bioluminescence measurements are reported in relative light units (RLU). Suitable detectors comprise a photomultiplier tube (PMT), a charge-coupled device (CCD), and/or any other luminometer.

In particular embodiments, the luminescent measurement is conveyed to a computer or other display means and/or storage means to illustrate the result to a user and/or store the result.

In various alternative embodiments, tubing and syringe pumps are then used to inject sample and/or reagent fluids through the channels at a precise rate or rates.

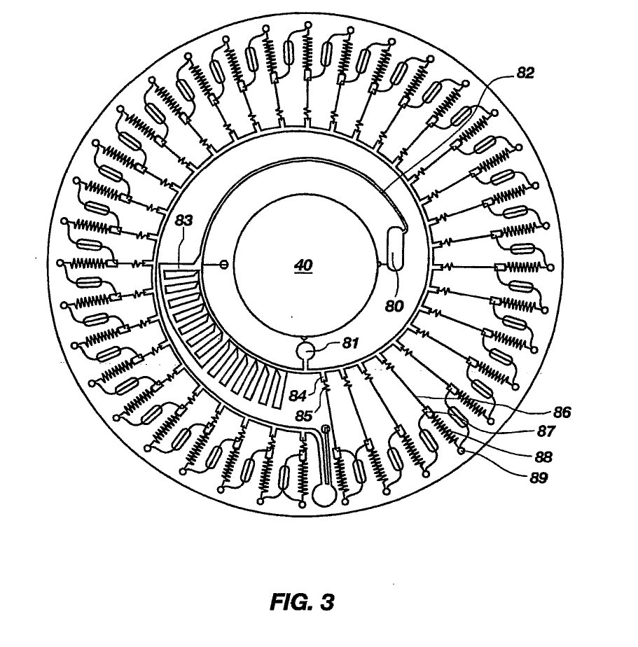

Now referring to FIG. 3, a view of FIG. 1 from a perspective above platform 1, a multi-analyte capable platform is illustrated. In an embodiment of operation, a sample is added to input sample well 80. Motor 40 rotates the embodiment in a clockwise fashion. The sample begins to travel along line 82. The frequency of the motor will directly affect the rate or speed at which the sample travels. In various embodiments, decanter(s) 83 are used to allow separation of the sample, such as blood and plasma. The sample then travels to sample collection well 84. In various embodiments, well 84 has sloped surfaces to assist the sample in traveling. In yet further embodiments, hydrophobicity can be used. Alternately, a sample is added to alternate sample well 81.

Sample metering valve 85 at least partially controls the flow of the sample into reagent well 87. In various embodiments, valve 85 is a narrow portion of the line, is in a zig-zag orientation, is hydrophobic, and/or the like. In particular embodiments, to move the sample beyond valve 85, the frequency is increased such that the sample bursts valve 85.

The sample then travels to reagent well 87 and begins the reaction. In particular embodiments, then frequency is further increased and the sample passes through reagent valve 88 and into reaction well 89. As the reaction occurs, a luminescence is generated that is detected by a photo detector, as herein before described. Reagent well 87 can include calibration solutions, initiating reagents, immunoassay compound or washing fluids initiate, calibrate, or otherwise prepare reactions in reaction well 89. As the reaction begins, the luminescent signal can be read from each well as it spins above a photodetector as seen in FIG. 2. The time intensity profiles for each well are recorded and used to calculate the concentration of the specific analyte of interest.

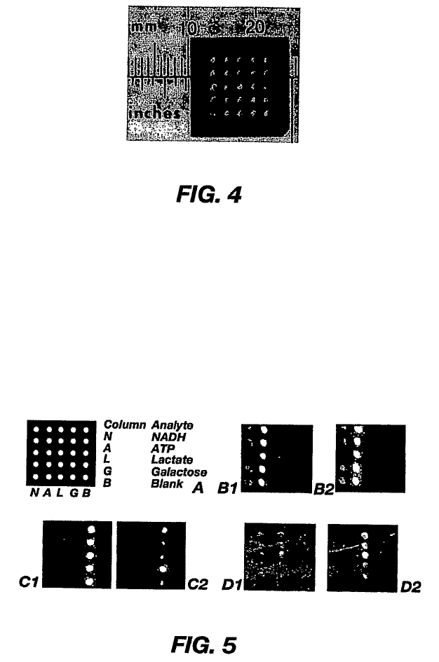

Now referring to FIG. 4, an alternative embodiment of the present invention, a non-rotating platform is disclosed. Luminescent experiments were performed on platform 50. In particular embodiments, platform 50 consisted of 5×5 arrays of 1 mm diameter holes, reaction well(s) 55, spaced 2 mm apart. Reaction well(s) 55 were cut in 15 mm squares out of matrix 60, 0.180 mm thick adhesive backed vinyl film with the Graphtec FC5100A-75 knife plotter (Graphtec). Platform 50 was then adhered to 15 mm square glass cover slips after manually removing the cut holes. The glass cover slips became the clear bottom for the 140 nL wells. Alternative embodiments were made by transferring multiple squares with the array of holes to clear polyester sheets at the same time and later cutting them to size.

Reference to FIG. 5 illustrates CCD images of luminescent platform arrays from an embodiment of FIG. 4. A) Bioluminescence assays were dispensed in separate columns for replicate data (5 rows per column). B1) NADH and ATP at 1 and 0.1 mM, respectively. B2) NADH and ATP at 0.01 and 0.001 mM, respectively. C1) Galactose assay (1 mM sample) at first 30 s exposure. C2) Galactose assay (1 mM sample) at sixth 30 s exposure. This competition luminescence dims with time. D1) Lactate assay (10 mM sample) at first 30 exposure. (Streaks of light across are due to a cracked cover slip.) D2) Lactate assay (10 mM sample) at sixth 30 s exposure. A photo detector measured the resulting luminescence.

In various embodiments, platforms are calibrated. Calibration means may be included on the platform as an on-board calibration means, calibration system(s), and/or a calibration sample. In particular embodiments, an on-board calibration could be performed by loading a known amount of analyte in a reaction chamber. The addition of sample to the reaction chamber and the resulting photo signal can be used to calibrate the device and/or establish a calibration curve. In various embodiments, an on-board calibration is used to standardize and/or normalize variables that affect measurements, such as, but not limited to storage time, variation between batches, interference effects, impurities in the sample, and/or the like. As well, such on-board calibration may be a factor in seeking and acquiring regulatory approval. Further, each optical detector, or transducer, in the detector arrays can be calibrated and tested for stability under varying conditions such as operating temperature bias voltage.

There are a variety of suitable dispensing systems for various embodiments of the present invention. In particular embodiments, the system should be capable of dispensing a sample or reagent volume less than 1 μL. Various embodiments of such systems use a variety of contact and non-contact printing technologies. Types of contact include pin printing, microcontact printing, discontinuous dewetting, gel patterning and screen printing. Pin printing and micro contact printing work by touching a pointed tip, wetted with the sample to be deposited, onto a hydrophilic surface. The sample then remains on the substrate. Pin printing is used often for printing DNA probes and self assembled monolayers. Microcontact printing can have 40 nm accuracy. Discontinuous dewetting is similar to pin printing but uses hydrophobic wells as the substrate. Gel patterning and screen printing are used for mass production and have been used for patterning enzyme-based sensors. Some of the commercially available contact printing systems are available from Affymetrix, (Santa Clara, Calif.), Cartesian Technologies Inc. (Durham, N.C.), SpotArray from Packard Biochip Technologies LLC (Billerica, Mass.), and GeneMachines (San Carlos, Calif.).

Non-contact printing is sometime known as drop on demand. Much of the work in this are has been for ink-jet printing. Non-contact dispenser methods include thermal percolators (find ref), piezoelectric actuated, flow through, acoustic transfer, and pressurized solenoid systems. Thermal ink-jet printing dispenser would not work for this research because the heat would denature the bioluminescent enzymes and clog the nozzles.

Piezoelectric dispensers. Flow-through dispensers dispense fluids as it flows through a channel or tubing.

Embodiments of the present invention further comprise processes for measuring the luminescence of a sample in a point-of-care device. In particular embodiments, the process comprises the steps of:
introducing a sample to a platform of a point-of-care device;
rotating the platform to create centrifugal force;
contacting the sample with a reagent; and,
measuring luminescence through a portion of the platform.

In an embodiment of operation of the embodiment illustrated in FIG. 3, wherein the sample is whole blood, the following steps can be performed. In the embodiment of whole blood separation on an embodiment of as platform of the present invention, the CD spins/is rotated at about 60 Hz, separating hematocrit and plasma in decanter(s) 83. In this embodiment, a flexible membrane is sealing decanter(s) 83 and expands to fill with the entire whole blood sample as it separates. The CD is then slowed down to about 5 Hz, whereupon the flexible membrane sealing the decant chambers contracts and ejects the plasma into the main sample delivery channel 90. The CD is then sped up to about 20 Hz to force the ejected sample along channel 90. As the sample travels, it fills the collection well(s) 84. In various embodiments, well 84 has sloped surfaces to assist the sample in traveling. In yet further embodiments, channel 90 can be hydrophilic to accelerate sample delivery by capillary action in addition to centrifugal pumping. Alternately, a sample is added to alternate sample well 81.

Sample metering valve 85 controls the flow of the sample into reagent well 87. In various embodiments, valve 85 is a narrow portion of the line, is in a zig-zag orientation, is hydrophobic, and/or the like. In various embodiments, valve 85 is different than prior art passive valve(s) (which consist of short, narrow hydrophobic sections) in that it is capable of metering samples with low surface tension, such as plasma. In particular embodiments, to move the sample beyond valve 85, the frequency is increased such that the sample bursts valve 85. In particular embodiments, this first burst frequency is higher than the 20 Hz required to deliver/convey the sample along channel 90 and into the collection wells 84.

Various embodiments of the present invention may be configured for the measurement of a multitude of assays comprising blood parameters, hematocrit levels, immunoassays, and/or the like. Suitable assays comprise, but are not limited to, phenylalanine, glucose, glucose 6-phosphate, galactose, galactose-1-phosphate (G-1-P), lactose, lactate, pyruvate, creatine, and creatinine in solution, human blood (serum & plasma), and urine. Further embodiments are expected to function for bioluminescence and chemiluminescence assays beyond clinical chemistry, such as but not limited to chemiluminescent immunoassays {20} for measuring drugs and steroids. Generally, any metabolite that can be measured via the ATP and NADH bioluminescent-based platforms can be measured in an embodiment of the present invention. Further, essentially any metabolite that can be measured via the H2O2 chemiluminescent-based platform can be measured in an embodiment of the present invention. Also, prior art fluorescence-based assays can be implemented on the CD device presented provided legal licensing is obtained for the specific assays.

### EXAMPLES

### Fabrication of Embodiment of the Present Invention

An embodiment of a μTAS device of the present invention was microfabricated in an elastomer using “soft-lithography.” In an exemplary, non-limiting embodiment, bioluminescent detection assays for two model analyte solutions (galactose and lactate) will be stabilized in individual detection wells. An array of photo detectors was used to measure the luminescent signal from each analytical well. Sample delivery, rehydration and mixing where studied. Onboard calibration channels where cut into the platform. Blood and urine samples where tested.

The bioluminescent reagents were packaged in a stable form within the reaction wells without exposure to heat. Various microfabrication methods were tested for creating microfluidic and encapsulating them without the standard approached which involve heat and/or oxidation. The prototyping method also had to be convenient and rapid enough to be able to test a variety of sample delivery approaches. The first method used was soft-lithography, a microfabrication technique which molds microfluidic structures in poly(dimethylsiloxane) (PDMS). This method is widely used for prototyping microfluidic structures due to its low cost and design flexibility, in addition to material property benefits of PDMS. Most PDMS microfluidic devices are cast on photolithographically patterned SU-8, a positive photo resist for features up to 1 mm thick. The mold for the initial device was made from an epoxy cast of a “chips” design machined in Teflon. The platform was made by molding PDMS on the cast and bonded them to glass cover slides before and filling them with the bioluminescent reagents.

Another fabrication method tested was laser cutting holes in plastic. And adhering clear adhesive on the bottom.

### Sampled Delivery to Reaction Wells

Two methods were studied. The first method used a wicking membrane to spread the sample out across an array of wells, as illustrated in FIG. 5. The wells were cut in a single layer of adhesive backed polymer bound to glass cover slides. The wells were filled with the bioluminescent reagents and freeze dried, creating the platform. The wicking membrane was then glued to or clamped onto the platform with the well array. Sample volume delivered to the wells was not precisely controlled, but diffusion of reagents between wells was tested to determine if there were any cross-talk effects.

The second sample delivery method used centrifugal pumping to aliquot sample to individual reaction wells on a CD. Microfluidic channels and wells were cut in an adhesive backed polymer and bound to a clear polycarbonate CD. The wells were filled with reagents and lyophilized and then sealed with another layer of adhesive backed polymer. Additionally, blood separation structure was designed into the device as well, allowing only plasma to be delivered to the reaction wells. Rotational speed controlled the separation and sample delivery.

### Analytes Tested

Five assays were tested on the bioluminescence-based biosensor developed in this research. The five assays were creatinine, galactose, glucose, lactate, and phenylalanine.

### Creatinine

Serum creatinine measurements are used to assess kidney function and glomerular filtration rate (GFR) (Rupert). Normal adult serum creatinine levels range from 50 to 100 μM. Since creatine concentration is relatively constant, the measurement of creatine in urine is used to allow for correction of urine dilution when measuring other analytes in urine.

Creatine was measured via creatinine deaminase and the ATP platform reaction as seen here:

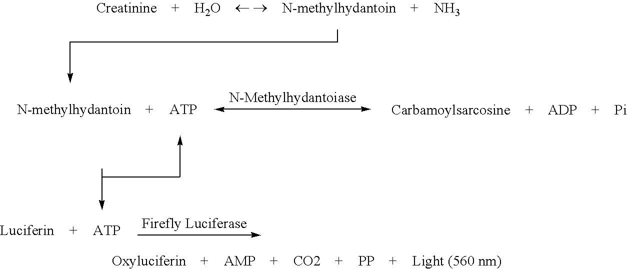

### Galactose

Galactose measurements are used in the management of galactosemia. Normal serum galactose concentration in newborns is 0-44 μM, while galacosemics can have galactose concentrations in the millimolar range.

Galactose was measured through the galactokinase and ATP platform reaction according to the following sequence:

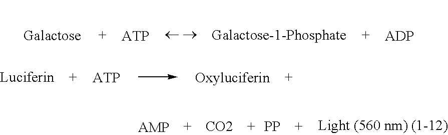

### Glucose

Glucose is a frequently measured analyte and is commonly measured to help diabetics monitor and manage their blood glucose levels through diet and insulin injections. Glucose concentrations in blood can range from 3 to 6 mM in normal patients and 5 to 20 mM in diabetics.

The glucose assay tested on the device consisted of the via glucokinase and ATP platform reaction below.

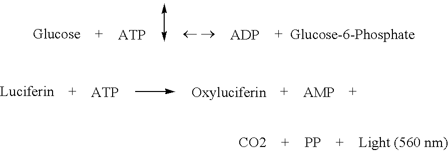

### Lactate

Lactate is a significant metabolite in the anaerobic glycolytic pathway. Increased lactate concentration in blood is an indicator of cellular oxygen deficiency as well as a marker of ischemia, hypoxia, and anoxia caused by variety of disorders, such as shock, respiratory failure, and congestive heart failure. Normal blood lactate concentrations are approximately 0.5 to 2.5 mM; lactate concentrations greater than 7 mM are cause for distress in sick patients. Lactate concentrations can increase in healthy patients during strenuous exercise and are used as an indicator of exercise intensity.

Lactate will be detected by the NADH bioluminescent platform according to the following sequence:

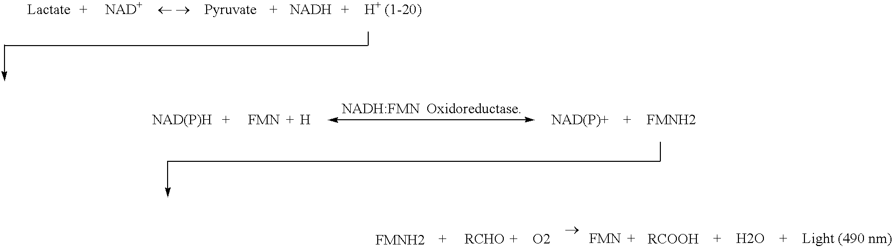

### Phenylalanine

Phenylketonuria (PKU) is a genetic deficiency which results from a defect in phenylalanine hydroxylase. It causes a chemical imbalance as well as an increase in phenylalanine concentrations in both serum and urine. Normal phenylalanine measurements range from 50 tp 150 μM but can go up to 500 μM for those with PKU. Measuring blood phenylalanine can help those with PKU manage their dietary intake of phenylalanine.

Phenylalanine is measured via phenylalanine dehydrogenase and the NADH bioluminescent platform as seen here:

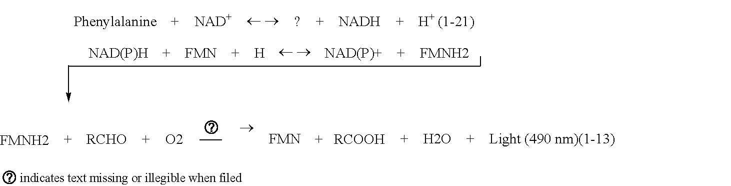

### Reagent Deposition and Configuration

A dispensing system using solenoid valves (available as INKX0516350AA, from The Lee Co., Westbrook, Conn.) capable of dispensing 40 to 500 nL droplets was built and tested for volume consistency. 1.97-inch long stainless steel nozzles (0.05 inch OD, 0.031 inch ID) fit with 0.005 inch (±0.0002 inch) orifices laser cut in sapphire (INZX0530450AA, The Lee Co.) were used to aspirate and dispense up to 24 μL of enzyme reagents without contaminating the solenoid's active parts. A spike and hold driver circuit (IECX0501350AA, The Lee Co.) was used to open and hold the solenoids for extended periods of time when aspirating and cleaning the nozzles, without over heating the solenoid. The 24 V spike was set to 90 microseconds, the shortest spike width required to open the solenoid. The holding voltage was set to 3.1 V, the lowest voltage required to hold the solenoid open. Pulse width and number of pulses were controlled by a National Instruments PCI 6601 counter card.

In order to aspirate and dispense multiple reagents, six miniature solenoid valves were plumbed to a computer controlled syringe pump (0162573 PSD/2, Hamilton Co. (Reno, Nev.)) fitted with an eight port valve and a 500 μL syringe. A LabView program was used to communicate with the PSD/2 via the computer's serial COM port. The pulses from the PCI 6601 counter card were directed to one of six spike and hold driver circuits by a 8 channel multiplexer (DG408DJ, Analog Devices (Norwood, Mass.)) which was controlled by TTL output signals from the PSD/2. The TTL outputs were controlled by serial commands from the LabView program. Before dispensing, a three way valve opened the solenoid line to an air line regulated to pressures ranging from 2 to 10 PSI.

### Reagent Deposition and Stabilization

The six micro-solenoid dispensers were attached to a vertical stepper motor translation stage (VT-80-25-2SM, Phytron, Inc. (Williston, Vt.)). The dispensing platform was attached to an XY stepper motor translation stage (VT-80-150-2SM, Phytron, Inc.). The translation stages were controlled by a 4-axis motion control card (PCI-7334, National Instruments) via a LabView program. Each stepper motor was powered by a microstepper motor driver which resulted in a 0.5 μm step per pulse.

The dispensing platform consisted of a 100×100×25 mm copper box inside a Delrin box. When dispensing, the substrates were placed on top of the copper box and were then filled with dry ice. At equilibrium, the substrates were less then −60° C., which caused the droplets to freeze within seconds of being dispensed. Rapid freezing prevented both evaporation of the reagent and denaturing of the enzymes.

After dispensing, the substrates were placed in the sample chamber of a VirTis Genesis 12 pilot plant lyophilizer, at a shelf temperature of −50° C. Primary lyophilization was performed at less than 100 mTorr with the condenser chamber cooled to −70° C. for 48-72 hours. Secondary lyophilization was then performed for 12-24 hours after changing the sample chamber to 25° C. at an average ramp rate of ˜3° C./hour. Lyophilized samples were stored in vacuum sealed bags with desiccant.

### Parallel Sample Delivery

Initial bioluminescent experiments were performed on platforms consisting of 5×5 arrays of 1 mm diameter holes spaced 2 mm apart. The holes were cut in 15 mm squares out of 0.180 mm thick adhesive backed vinyl film with the Graphtec FC5100A-75 knife plotter (Graphtec (Irvine, Calif.)). The array patterns were then adhered to 15 mm square glass cover slips after manually removing the cut holes. The glass cover slips became the clear bottom for the 140 nL wells (FIG. 3-1). Later, ChemChips were made by transferring multiple squares with the array of holes to clear polyester sheets at the same time and later cutting them to size.

Sample delivery on these platforms was achieved by clamping filter membranes above the wells on the center of the array. The sample wicked along the membrane and into each well, whereupon the reagents were dissolved and the bioluminescent reactions began. Since reagent drops were larger than the volume of the wells, a convex meniscus formed above each well. This convex structure, porous and hydrophilic in nature after lyophilization, facilitated drawing the sample from the membrane into each well without the risk of bubble formation.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

### REFERENCES

{1} P. -A. Auroux, D. Iossifidis, D. R. Reyes, and A. Manz, “Micro total analysis systems. 2. Analytical standard operations and applications,” Analytical Chemistry, vol. 74, pp. 2637-2652, 2002.

{2} D. R. Reyes, D. Iossifidis, P. -A. Auroux, and A. Manz, “Micro total analysis systems. 1. Introduction, theory, and technology,” Analytical Chemistry, vol. 74, pp. 2623-2636, 2002.

{3 } T. Vilkner, D. Janazek, and A. Manz, “Micro total analysis systems. Recent developments,” Analytical Chemistry, vol. 76, pp. 3373, 2004.

{4} I. Macfarlane and F. Davis, “Building blocks for the point-of-care boom,” in IVD Technology, 2002, pp. 27-34.

{5} T. H. Grove, “In vitro diagnostics: Bringing testing to the point of care,” in Medical Device & Diagnostic Industry, vol. April, 2000, pp. 78-94.

{6} A. J. Tudos, G. A. J. Besseling, and R. B. M. Schasfoort, “Trends in miniaturized total analysis systems for point-of-care testing in clinical chemistry,” Lab on a Chip, vol. 1, pp. 83, 2001.

{7} G. Wienhausen and M. DeLuca, “Bioluminescent assays of picomole levels of various metabolites using immobilized enzymes,” Anal Biochem, vol. 127, pp. 380-388, 1982.

{8} D. Min, R. Stewart, and J. Andrade, “Biosensor for phenylalanine based on bacterial bioluminescence,” in Bioluminescence and chemiluminescence: Perspectives for the 21st century, A. e. a. Roda, Ed.: Wiley, 1999, pp. 520-523.

{9} R. H. Davies, J. Eu, D. Min, and J. D. Andrade, “Bioluminescence assays for creatine and creatinine for biosensor applications,” presented at Biosensors Conference 2000, San Diego, 2000.

{10} K. Tsukagoshi, N. Jinno, and R. Nakajima, “Development of a micro total analysis system incorporating chemiluminescence detection and application to detection of cancer markers,” Analytical Chemistry, vol. 77, pp. 1684, 2005.

{11} Q. Fang, X.-T. Shi, Y.-Q. Sun, and Z.-L. Fang, “A flow injection microdialysis sampling chemiluminescence system for in vivo on-line monitoring of glucose and intravenous and subcutaneous tissue fluid microdialysates,” Analytical Chemistry, vol. 69, pp. 3750-3577, 1997.

{12} A. Economou, D. Papargyris, and J. Stratis, “Automated flow-injection instrument for chemiluminescence detection using a low-cost photodiode detector,” Journal of Chemical Education, vol. 81, pp. 406, 2004.

{13} Schembri, C. T.; Ostoich, V.; Lingane, P. J.; Burd, T. L.; Buhl, S. N. Clin. Chem. 1992, 38, 1665-1670.

{14} Schembri, C. T.; Burd, T. L.; Kopfsill, A. R.; Shea, L. R.; Braynin, B. J. Autom. Chem. 1995, 17, 99-104.

{15} Scott, C. S.; Burtis, C. A. Anal. Chem. 1973, 45, 327A-340A. Scott, C. S.; Burtis, C. A. Centrifugal Analysis in Clinical Chemistry; Praeger: New York, 1980.

{16} Madou, M.; Kellogg, G. J. Proc. SPIE 1998, 3259, 80-93.

{17} “Point-of-Care Analyzers, Clinical Laboratory,” Healthcare Product Comparison System (Plymouth Meeting, PA: ECRI, 2001), 1-60.

{18} M. A. Schwarz and P. C. Hauser, Lab Chip, 2001, 1, 1-6.

{19} Y. Xu, F. G. Bessoth, J. C. T. Eijkel, and A. Manz, “On-line monitoring of chromium(iii) using a fast micromachined mixer/reactor and chemiluminescence detection,” in The Royal Society of Chemistry, vol. 125, 2000, pp. 677-683.

{20} X. Yang, J. Janatova, and J. D. Andrade, “Homogeneous enzyme immunoassay modified for application to luminescence-based biosensors,” Anal Biochem, vol. 336, pp. 102-107, 2005.

{21} T. Brenner, S. Haeberle, R. Zengerle, and J. Ducree, “Continuous centrifugal separation of whole blood on a disk,” presented at International Conference on Miniaturized Systems for Chemistry and Life Sciences, Malmö, Sweden, 2004.

{22} D. C. Duffy, H. L. Gillis, J. Lin, N. F. Sheppard, Jr., and G. J. Kellog, “Microfabricated centrifugal microfluidic systems: Characterization and multiple enzymatic assays,” Analytical Chemistry, vol. 71, pp. 4669-4678, 1999.

What is claimed is:

1. A system for point-of-care diagnostic measurements comprising:
a platform comprising a matrix,
a luminescent reagent,
a means to rotate the platform, and
a photo detector,
wherein the matrix has been formed to create a reaction well and wherein introduction of a sample into the reaction well allows for a luminescent reaction with the luminescent reagent, wherein said luminescent reaction is detected by the photo detector.

2. The system of claim 1, wherein the platform comprises a compact disk, and wherein the compact disk is rotated.

3. The system of claim 1, further comprising a metal coating.

4. The system of claim 1, further comprising a decanter.

5. A bioluminescent system for point-of-care diagnostic measurements comprising:
a matrix,
a motor,
a luminescent reagent, and
a detector,
wherein the motor rotates the matrix such that a sample associated with the matrix contacts the luminescent reagent producing a signal that is measured by the detector.

6. The bioluminescent system of claim 5, wherein the matrix further comprises a channel formed in the matrix.

7. The bioluminescent system of claim 5 wherein the sample is blood.

8. A process for measuring the luminescence of a sample in a point-of-care device, said process comprising:
introducing a sample to a platform of a point-of-care device;
rotating the platform to create centrifugal force therein;
contacting the sample with a reagent; and
measuring luminescence through a portion of the platform.

9. The process of claim 8, wherein the platform comprises a channel.

10. The process of claim 8, wherein multiple samples are introduced to the platform for multiple measurements.

11. The process of claim 8, wherein the luminescence is measured and/or detected with a photo detector.

12. The process of claim 8, wherein the sample comprises blood or plasma.

13. The process of claim 8, wherein the channel further comprises a reagent well.

14. The process of claim 8, further comprising the step of preparing samples by centrifugal separation.
